(19) United States
(12) Patent Application Publication
(10) Pub. No.: US 2016/0302719 A1
(43) Pub. Date: Oct. 20, 2016

(54) REMOTE LOW BLOOD OXYGEN LEVEL WARNING SYSTEM AND RELATED METHOD

(71) Applicant: Collins Ezeuka, Calabasas, CA (US)

(72) Inventor: Collins Ezeuka, Calabasas, CA (US)

(21) Appl. No.: 14/690,272

(22) Filed: Apr. 17, 2015

(51) Int. Cl. A61B 5/00; A61B 5/1455

(52) U.S. Cl. CPC A61B 5/4818; A61B 5/0004; A61B 5/7246; A61B 5/7282; A61B 5/746; A61B 5/6806; A61B 5/6803; A61B 5/6807; A61B 5/14552; A61B 5/7405; A61B 5/742; A61B 2503/04

(57) ABSTRACT

The low blood oxygen level warning system generally includes a sensor for measuring a plurality of oxygen saturation levels in, e.g., an infant. A communication circuit transmits a signal associated with at least one of the plurality of oxygen saturation levels measured by the sensor to a remote device. A controller compares at least one of the plurality of oxygen saturation levels measured by the sensor to a predetermined threshold oxygen saturation level. The remote device provides an alarm in response to an undesired oxygen saturation level of which is relatively lower than the predetermined threshold oxygen saturation level.

### BACKGROUND OF THE INVENTION

The present invention generally relates to a remote low blood oxygen level warning system and related method for operating the same. More specifically, the present invention relates to a system that includes a sensor for measuring the blood oxygen level of a wearer at select intervals and generates a remote notification when the blood oxygen level drops below some predetermined threshold level so the wearer can receive immediate emergency care.

Sudden Infant Death Syndrome (“SIDS”) is the sudden death of a newborn up until about age one when the cause of death cannot be explained after a thorough investigation regarding the circumstances of the death. SIDS is one of the leading causes of death among infants between one month and twelve months, and can claim between 2,500-3,500 lives each year in the United States. During SIDS, the infant suffers from asphyxiation during sleep, which usually occurs without the infant making any noise. This can result from suffocation by soft bedding (e.g., pillow or waterbed mattress), overlay (e.g., one person rolls against or over the infant while sleeping), wedging or entrapment (e.g., the infant wedges between two objects such as a mattress and wall or bed frame), or strangulation. As a result, SIDS is a silent killer and otherwise extremely difficult to detect. The Centers for Disease Control and Prevention (“CDC”) provides recommendations to parents and caregivers for reducing the health risks associated with SIDS, such as placing the baby on its back to sleep, using a firm sleep surface and safety-approved cribs, sharing a room with the baby (but not the bed), and keeping soft objects like pillows and loose bedding away from the sleeping area. But, SIDS can occur without warning with respect to seemingly healthy babies, and even despite following the CDC's recommendations. As a result, even taking the precautions recommended by the CDC, a parent or caregiver may be unaware that an infant is still suffering from SIDS (e.g., the baby may roll over when sleeping). As such, the parent or caregiver may be unable to timely change the sleeping position of the infant, provide life-saving resuscitation, or contact emergency medical services in the event of an emergency.

There exists, therefore, a significant need for a remote low blood oxygen level warning system that includes a sensor for measuring the blood oxygen level of a wearer, an integrated communication circuit for sending a signal to a remote device, comparing the blood oxygen level at select intervals against a predetermined threshold oxygen saturation level, and an associated warning system for alerting a third party caregiver that the blood oxygen level of the wearer has dropped to an undesired level. The present invention fulfills these needs and provides further related advantages.

### SUMMARY OF THE INVENTION

The low blood oxygen level warning system as disclosed herein generally includes a sensor for measuring a plurality of oxygen saturation levels in, e.g., an infant. A communication circuit transmits a signal associated with at least one of the plurality of oxygen saturation levels measured by the sensor to a remote device. A controller compares at least one of the plurality of oxygen saturation levels measured by the sensor to a predetermined threshold oxygen saturation level. The remote device provides an alarm in response to an undesired oxygen saturation level of which is relatively lower than the predetermined threshold oxygen saturation level. The sensor is preferably a pulse oximetry sensor.

In one embodiment, the sensor removably attaches to a garment, such as a pair of footed pajamas, a glove, or a hat. Alternatively, the sensor may be mounted to or integrated into the garment. Preferably, the communication circuit is a wireless transmitter, and the signal transmitted thereby may be a single alarm signal or data related to a stream of oxygen saturation levels. In one embodiment, the controller couples to the sensor and operates the communication circuit to transmit the signal in response to identifying the undesired oxygen saturation level. Alternatively, the controller may couple to the remote device and receive the plurality of oxygen saturation levels from the communication circuit via the signal. Here, the communication circuit may couple directly to the sensor. The remote device, such as a Smartphone, a tablet, or an alarm, may produce an audible or visual alarm in response to reading an unsafe oxygen saturation level.

In an alternate embodiment, the low blood oxygen level warning system includes a garment-integrated sensor for measuring the plurality of oxygen saturation levels. A wireless transmitter transmits a signal associated with at least one of the plurality of oxygen saturation levels measured by the garment-integrated sensor. The signal may be a single alarm signal or data related to a stream of oxygen saturation levels. A microcontroller coupled to the garment-integrated sensor compares the plurality of oxygen saturation levels measured thereby to a predetermined threshold oxygen saturation level. The microcontroller operates the wireless transmitter to transmit the signal in response to identifying the undesired oxygen saturation level relatively lower than the predetermined threshold oxygen saturation level to the remote device, which provides an alarm in response to the undesired oxygen saturation level. Preferably, the garment-integrated sensor is a pulse oximetry sensor and the garment may be a pair of footed pajamas, a glove, or a hat. The remote device may be a Smartphone, a tablet, or an alarm, and alarm may be audible or visual.

The method for activating the alarm in response to an undesired oxygen saturation level in, e.g., the infant, generally includes reading a plurality of oxygen saturation levels with a sensor and comparing each of the plurality oxygen saturation levels read by the sensor to a predetermined threshold oxygen saturation level. The next step is to transmit a signal associated with at least one of the plurality oxygen saturation levels read by the sensor to a remote device over a communication network (e.g., a wireless communication network). The system then determines whether at least one of the plurality of oxygen saturation levels is undesired, i.e., relatively lower than a predetermined threshold oxygen saturation level. The system may then activate an alarm in response to determining that one of the plurality of oxygen saturation levels is undesired.

In one embodiment, the system may transmit the warning signal to the remote device over the communication network. Alternatively, the system may stream the plurality oxygen saturation levels to the remote device at periodic intervals or in real-time. Additionally, the system may sync the alarm across multiple remote devices, such as a tablet or infant monitoring system. The method as disclosed herein may optionally include removably attaching the sensor to a garment and sliding the sensor over an appendage of the user (e.g., a toe, a finger, or an ear lobe). The system may also include controls that allow the predetermined threshold oxygen saturation level that trigger the alarm to be adjustable.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a schematic view of one embodiment of the remote low blood oxygen level warning system as disclosed herein;

FIG. 2 is a diagrammatic view of the remote low blood oxygen level warning system of FIG. 1, further illustrating general communication between an infant and an adult;

FIG. 3 is a cross-sectional view of a toe-mounted garment integrated sensor taken generally about line 3-3 in FIG. 2;

FIG. 4 is an enlarged cross-sectional view similar to that taken about the circle 4 in FIG. 3, illustrating an alternative wherein the sensor removably attaches to the garment;

FIG. 5 is a schematic view of a preferred embodiment of the operational components of the sensor as disclosed herein;

FIG. 6 is an environmental view of the remote low blood oxygen level warning system, further illustrating transmission of a warning system through a home;

FIG. 7 is a flow chart illustrating a method for using the remote low blood oxygen level warning system in accordance with one embodiment disclosed herein; and

FIG. 8 is a flow chart illustrating steps for determining the oxygen saturation in the blood of a user.

### DETAILED DESCRIPTION OF THE PREFERRED EMBODIMENTS

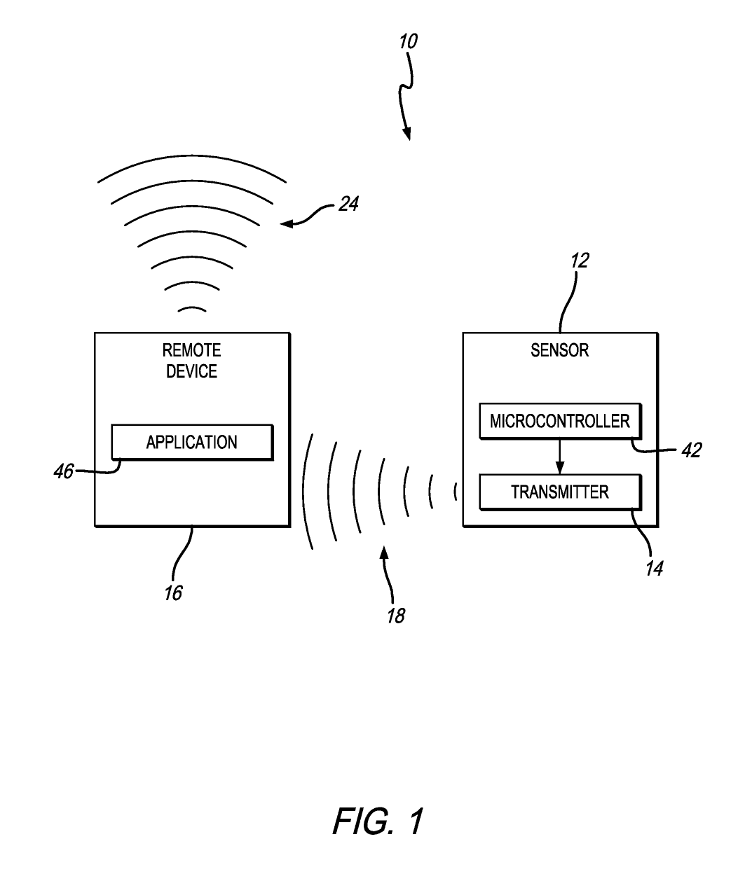

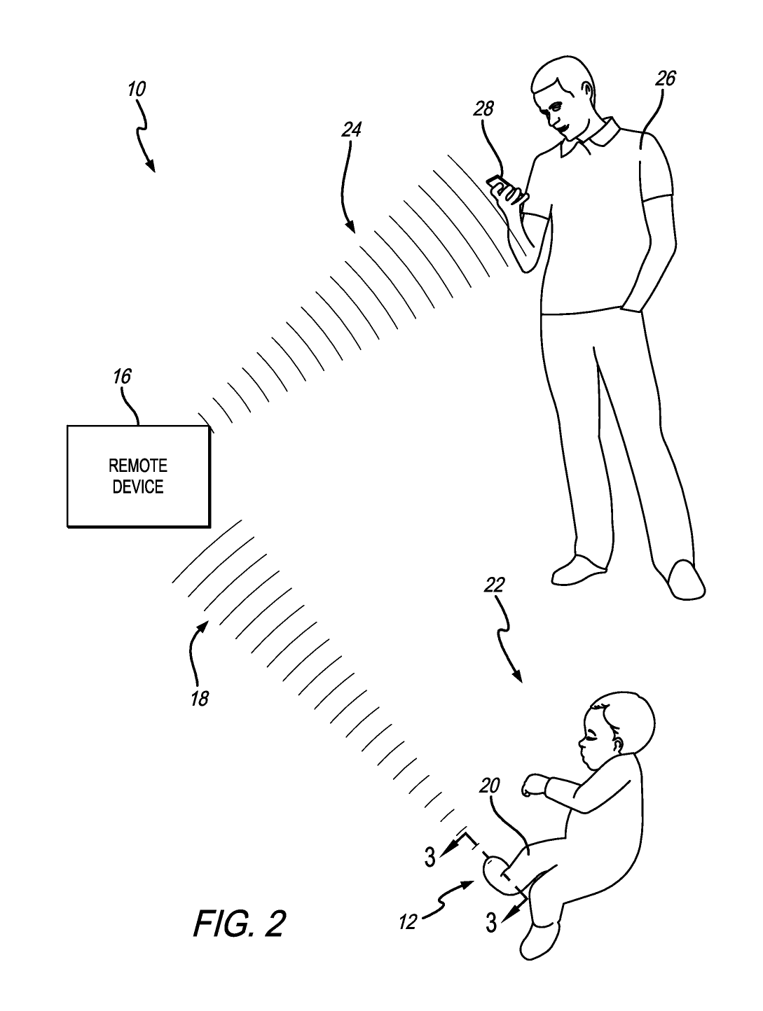

As shown in the drawings for the purposes of illustration, the present disclosure for a remote low blood oxygen level warning system is referred to generally by the reference numeral 10 in FIGS. 1 and 2. The system 10 generally includes a sensor 12 having a transmitter 14 that communicates with a remote device 16 over a communication network 18, such as by way of a wireless signal 18 as shown in FIG. 1. The sensor 12 is preferably a pulse oximeter, but may include any sensor known in the art for measuring the oxygen content (i.e., oxygen saturation) in the blood, and preferably at select intervals or in real-time. In one embodiment as illustrated in FIG. 2, the sensor 12 may be integrated into or otherwise attached to a garment 20 and used specifically to detect or monitor the oxygen saturation in the blood of an infant 22. If the oxygen saturation of the infant 22 falls below a predetermined threshold level, the sensor 12 may send the wireless signal 18 to the remote device 16, which then provides a notification or other warning 24 to an adult or caregiver 26 (e.g., to a cell phone or other portable electronic device 28) that the infant 22 has undesirably or dangerously low oxygen saturation. An acute drop in oxygen saturation below the threshold level may be an indication that the infant 22 is suffocating, has a foreign body lodged in the trachea (i.e., air way), is choking, or is otherwise experiencing a health problem that reduces the amount of oxygen in the blood (e.g., SIDS) and could potentially lead to asphyxiation and even death. In this respect, the warning 24 may provide an early indication that the infant 22 is experiencing such a health problem, thereby allowing the caregiver 26 to quickly seek or provide potentially life-saving medical assistance (e.g., resuscitation).

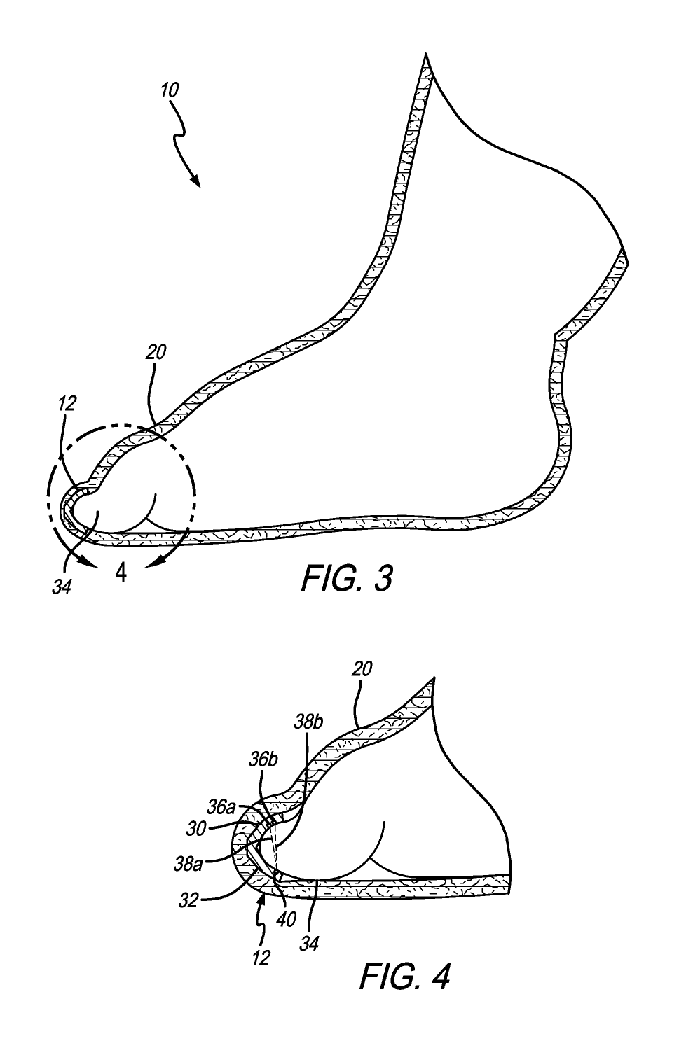

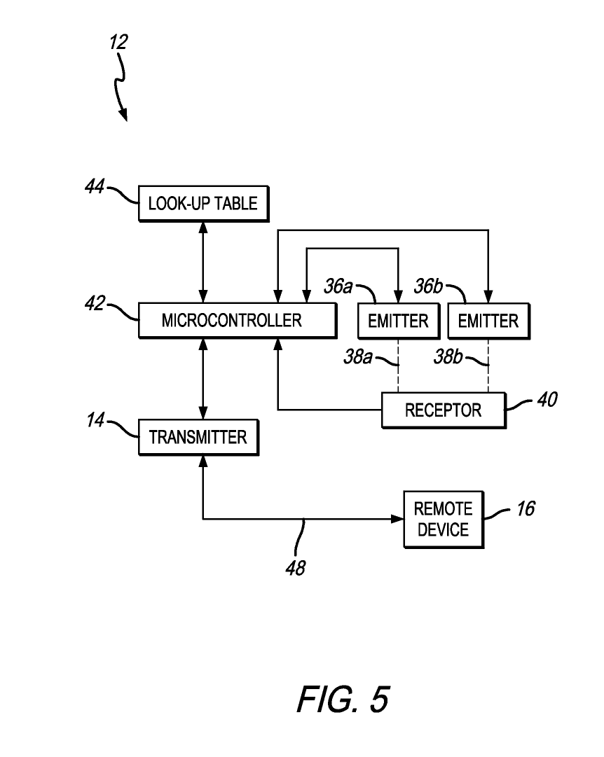

In a preferred embodiment, for example, FIGS. 3 and 4 illustrate that the sensor 12 is a generally clip-like structure that includes an upper member 30 pivotably connected to a lower member 32 that slides over a bodily appendage 34 (shown on a big toe) of the infant 22. Preferably, the sensor 12 is versatile and may be configured to attach to one or more body parts such as the toes, fingers, ear lobes, etc. The upper member 30 may include a pair of emitters 36a, 36b that each emit a corresponding light beam 38a, 38b into the appendage 34 for reception by at least one receptor 40 disposed in the lower member 32. Of course, the pair of emitters 36a, 36b may be disposed on the lower member 32 and the receptor 40 may be disposed on the upper member 30. In either embodiment, the pair of emitters 36a, 36b should be generally aligned with the receptor 40 when the sensor 12 is attached to the appendage 34. The pair of emitters 36a, 36b are preferably LEDs, but may be any suitable light source (e.g., incandescent lights) known in the art and capable of being used to measure oxygen saturation in the blood. One receptor 40 may correspond to one emitter 36, one receptor 40 may correspond to multiple emitters 36, and multiple receptors 40 may correspond to multiple emitters 36. In one alternative embodiment, the sensor 12 may be annular and, thus, fail to include distinct upper and lower members 30, 32.

In a preferred embodiment, a first emitter 36a emits an infrared light beam 38a and a second emitter 36b emits a red light beam 38b for reception by the single receptor 40. The oxygen level in the blood of the infant 22 affects the intensity of the light beam 38 received by the receptor 40. The microcontroller 42 may determine the oxygen saturation of the blood based on the loss in intensity of the light beam(s) 38 passing through the appendage 34 (e.g., toe, finger, ear lobe, etc.) of the infant 22. More specifically, oxygen attaches to hemoglobin, a protein inside of red blood cells, upon entering the blood of the infant 22. The blood then carries the oxygen to various tissues in the body of the infant 22 for consumption thereof. Oxygenated hemoglobin absorbs more infrared light than deoxygenated hemoglobin. Thus, the presence of oxygen reduces the intensity of infrared light. Conversely, deoxygenated hemoglobin absorbs more red light than oxygenated hemoglobin. As such, the lack of oxygen reduces the intensity of red light. In a preferred embodiment, the microcontroller 42 may measure the intensities of the infrared and red light beams 38a, 38b received by the receptor 40 and then compare the same to the intensities of the infrared and red light beams 38a, 38b respectively emitted by the first and second emitters 36a, 36b. The microcontroller 42 may then reference a look-up table 44 to determine the oxygen saturation level in the blood of the infant 22 based on the intensity losses in the infrared and red light beams 38a, 38b. If the oxygen saturation level is below the predetermined low blood oxygen level threshold, the microcontroller 42 may activate the transmitter 14 to generate the wireless signal 18 to the remote device 16 indicating that the oxygen saturation level is below the predetermined (e.g., desired) level.

In an alternative embodiment, the microcontroller 42 may communicate oxygen saturation levels to the transmitter 14 at select intervals or in real-time. These periodic oxygen saturation level readings may then be streamed to the remote device 16 over the communication network or wireless signal 18. An application 46 running in connection or otherwise associated with the remote device 16 may compare the periodic oxygen saturation levels against the predetermined threshold oxygen saturation level to determine whether to generate the warning 24 based on those readings.

In one embodiment, the sensor 12 may be advantageously integrated into the garment 20 as illustrated in FIGS. 3 and 4. In this embodiment, the sensor 12 may be woven or stitched into the fabric of the garment 20, as best shown in enlarged FIG. 4, or otherwise non-removably attached to the garment 20. Alternatively, the sensor 12 may removably attach to the garment 20 via, e.g., snaps or hook and loop fasteners (e.g., Velcro® brand fasteners sold by Velcro USA Inc. of 406 Brown Ave., Manchester, N.H. 03103) to facilitate removal thereof prior to, e.g., laundering the garment 20. Although, any other suitable attachment method (e.g., adhesive, thread, stitching, etc.) may also be used.

As such, the garment 20 preferably comfortably secures the sensor 12 to the appendage 34 of the infant 22 (e.g., a large toe shown in FIGS. 3-4) and prevents the sensor 12 from disengaging the infant 22 in the event the infant 22 moves (e.g., rolling over in a bed, walking, etc.) while the garment 30 is being worn. Obviously, the particular appendage to which the sensor 12 attaches determines the type of garment into which the sensor 12 integrates. In this respect, the sensor 12 may integrate into socks, shoes, footed pajama pants if the sensor 12 fits over a toe. For example, FIGS. 3 and 4 illustrate one preferred embodiment wherein the sensor 12 is integrated into a pair of footed pajamas 20a. Conversely, the sensor 12 may integrate into a glove if the sensor 12 fits over a finger or a hat if the sensor 12 fits over an ear lobe.

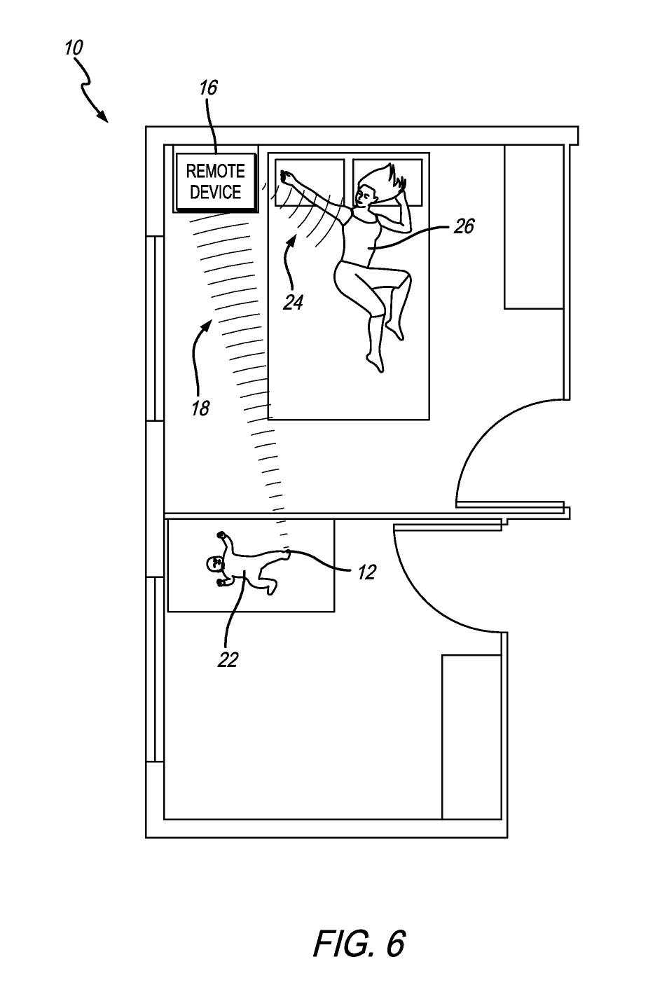

As briefly mentioned above and illustrated in FIG. 1, in one embodiment, the transmitter 14 may transmit the wireless signal 18 to the remote device 16 if the blood oxygen level in the infant 22 falls below a predetermined level. In this respect, the transmitter 14 preferably transmits the signal 18 wirelessly and more preferably transmits the signal 18 via Wi-Fi or Bluetooth. Although, other wired (e.g., LAN) or wireless (e.g., cellular network) communications networks 18 may be used to transmit the signal, as needed or desired. Of course, multiple communications networks (including both wired and wireless) could be utilized simultaneously, depending on the desired destination of the warning 24. In one embodiment, the remote device 16 may be a portable electronic device such as a Smartphone, tablet, laptop computer, watch, baby monitor, etc. In other embodiments, the remote device 16 may be a non-portable electronic device such as a television, built-in monitoring system, desktop computer, monitor, etc. Although, in general, the transmitter 14 on the sensor 12 should be able to establish a connection with the communication network 18 to facilitate unilateral or bi-lateral voice or data communication or the like, with the remote device 16. Preferably, as shown in FIG. 6, the remote device 16 is able to communicate with the sensor 12 over the communication network 18 when placed in a different room from the sensor 12. In this respect, the remote device 16 and the sensor 12 are preferably separate components.

Information received by the remote device 16 by way of the communication network 18 or the like is preferably processed locally by the application 46 or other like program installed onto the remote device 16 or otherwise operating in connection with the remote device 16. In one embodiment, the application 46 may process a single signal from the transmitter 14 indicating that the oxygen saturation of the infant 22 is already below the desired level. In this case, the remote device 16 may immediately produce the warning 24. Alternatively, the application 46 may process a stream of data conveyed by the transmitter 14 and process the data locally, such as comparing oxygen saturation levels to a threshold oxygen saturation level to determine whether to activate the warning 24. This feature may be particularly useful and helpful for recording data longer-term, especially in hospital settings. The warning 24 itself may be an audible noise (e.g., an alarm), a vibration (e.g., vibrate mode on a Smartphone or tablet), a text message, an email, a phone call, or any other suitable notification known in the art. The warning 24 notifies the caregiver 26 (e.g., a nurse, parent, friend, etc.) that the infant 22 has an oxygen saturation level that is below some predetermined threshold and is otherwise in possible danger of asphyxiation. As such, the caregiver 26 can provide or seek life-saving medical attention (e.g., resuscitation) to correct the low oxygen saturation in the infant 22.

The application 46 preferably permits the caregiver 26 to adjust and control the various parameters of the system 10. For example, the application 46 may permit the caregiver 26 to adjust the low blood oxygen content threshold, change the notification method (e.g., from vibration to alarm), or the characteristics of the notification (e.g., the volume of an audible alarm). Although, the application 46 may control any parameter or setting in the system 10 as needed or desired. Furthermore, the application 46 may install on multiple remote devices 20, thereby synchronizing the sensor 12 across multiple devices (e.g., a baby monitor and a Smartphone). In this respect, multiple remote devices 20 may produce the same or different warning(s) 24 should the infant 22 suffer relatively low oxygen saturation in the blood.

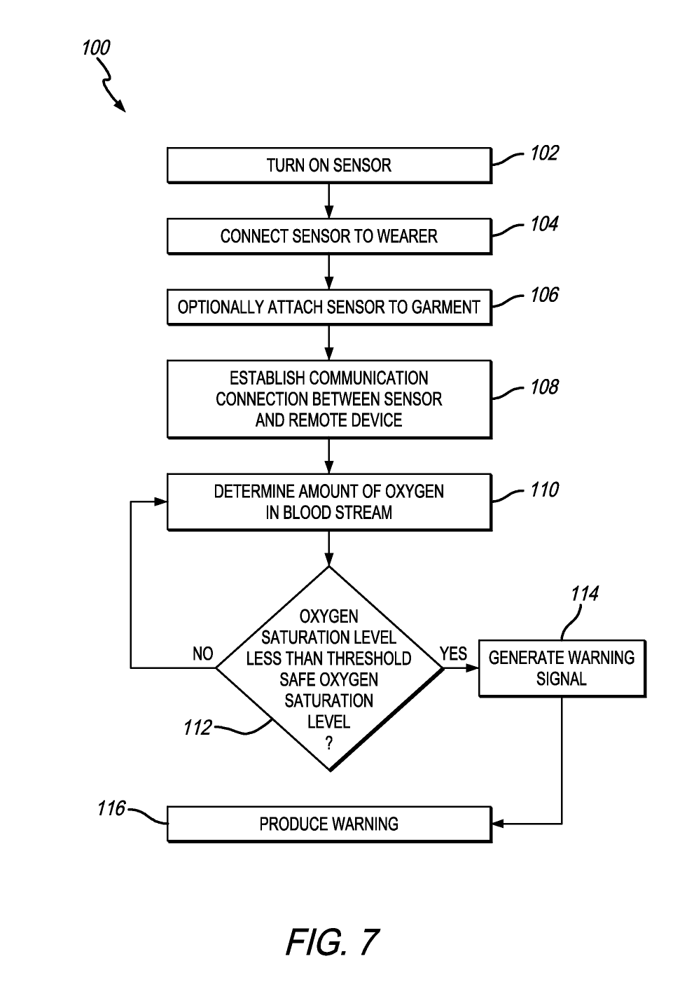

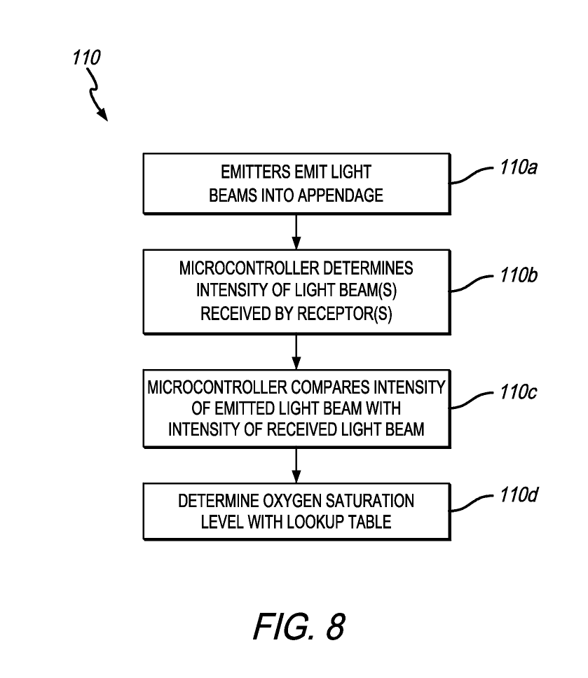

In a preferred embodiment illustrated in FIG. 7, the system 10 monitors the oxygen saturation in the infant 22, especially while sleeping, to warn the caregiver 26 (e.g., a parent) that the infant 22 may be suffering from SIDS. In the preferred embodiment, the sensor 12 fits over the large toe of the infant 22 to monitor the oxygen saturation in the blood. In this respect, the sensor 12 may be integrated into the foot portion of the footed pajamas, as mentioned above and shown in FIGS. 3 and 4. Advantageously, the pajamas retain the sensor 12 in the desired position (i.e., engaged with the large toe of the infant 22) even if the infant 22 moves while sleeping. As such, the infant 22 cannot easily dislodge or knock the sensor 12 off the toe because the foot portion of the pajamas secures the sensor 12 thereto. Should the oxygen saturation in the blood of the infant 22 fall below the threshold, the system 10 alerts the caregiver 26 in accordance with the embodiments disclosed herein. In the unlikely event the sensor 12 falls off the infant 22, the oxygen saturation level would quickly fall to zero, which may cause the system 10 to alert the caregiver 26 with the same or similar warning 24 as if the oxygen saturation level fell below the threshold level mentioned above. The difference here is that the level would immediately fall to zero as opposed to an undesired level below the threshold but above zero.

While the embodiments have been disclosed herein for use in connection with an infant 22, persons of ordinary skill in the art will readily recognize that the system 10 may be employed by users of any age (e.g., the elderly) or having any health condition that results or could result in low oxygen saturation in the blood (e.g., asthma, emphysema, sleep apnea, etc.).

FIG. 7 illustrates one method (100) for using the remote low blood oxygen level warning system 10 in accordance with the embodiments disclosed herein. In this respect, the first step (102) is to turn “on” the sensor 12. Powering “on” the sensor 12 activates the microcontroller 42 and other features operated thereby, such as the emitter(s) 36 and the receptor(s) 40, as described herein. The next step (104) is to attach the sensor 12 to the wearer (e.g., over the large toe of the infant 22) so the sensor 12 can start taking oxygen saturation readings. Optionally, in step (106), the sensor 12 may be attached to the garment 20 via, e.g., hook and loop fasteners, if the sensor 12 is not already integrated into the garment 20. The sensor 12 then establishes a communications connection with the remote device 16 over, e.g., the communication network 18 as part of step (108). This allows the sensor 12 and the remote device 16 to communicate, either unilaterally or bi-laterally, for monitoring the oxygen saturation in the infant 22, and importantly producing the warning 24 if the level falls below some predetermined threshold.

In step (110), the sensor 12 determines the amount of oxygen in the blood of the infant 22. As more specifically illustrated in FIG. 9, the emitter(s) 36 emit the light beam(s) 38 into the appendage 34 as part of step (110a). For example, in a preferred embodiment, the first emitter 36a emits the infrared light beam 38a and the second emitter 36b emits the red light beam 38b. In step (110b), the microcontroller 42 determines the intensity of the light beam(s) 38 received by the receptor(s) 40 after passing through the appendage 34. Next, the microcontroller 42 compares the difference in intensity of the light beam(s) 38 between the emitter(s) 36 and the receptor(s) 40, as part of step (110c). Then, as part of step (110d), the microcontroller 42 uses the look-up table 44 to determine the oxygen saturation in the blood of the infant 22 based on the loss in intensity.

In step (112), the microcontroller 42 compares the oxygen saturation level in the blood of the infant 22 calculated during step (110d) to the predetermined low blood oxygen level threshold. If the oxygen saturation level in the infant 22 is below the threshold level, the warning 24 may be generated (114). Alternatively, if the oxygen saturation level is above the threshold, the sensor 12 measures the oxygen saturation level again in accordance with step (110). The loop between steps (110) and (112) repeats while the oxygen saturation levels are acceptable (i.e., above some predetermined threshold level).

With respect to step (114), in one embodiment, the microcontroller 42 may cause the transmitter 14 to generate a wireless signal 18 to the remote device 16 indicating that the oxygen saturation is too low. Alternatively, step (114) may be accomplished by the remote device 16, namely the application 46 may determine that the streamed or periodic oxygen saturation levels transmitted by the transmitter 14 have fallen below the desired threshold level. In this embodiment, the application 46 activates the warning 24, not the sensor 12. The system 10 preferably sends the warning 24 that the infant 22 has undesirably low blood oxygen levels to notify the caregiver 26. In this respect, the system 10 is able to alert the caregiver 26 in real-time and in virtually any location (e.g., a different room, building, etc.) that the infant 22 is in danger of asphyxiation. That is, the system 10 provides a remote warning to a caregiver that may otherwise be unaware that the infant 22 is suffering a medical condition that could potentially lead to asphyxiation and death. In one such embodiment illustrated in FIG. 6, this may occur in a situation where a parent 26 is in one room of a house while the infant 22 sleeps in another room. As such, the parent 26 can rush to the infant 22 to remedy the situation, e.g., adjust the infant's sleeping position, resuscitate the infant 22, and/or call for emergency medical assistance.

Although several embodiments have been described in detail for purposes of illustration, various modifications may be made without departing from the scope and spirit of the invention. Accordingly, the invention is not to be limited, except as by the appended claims.

What is claimed is:

1. A low blood oxygen level warning system, comprising:
a sensor for measuring a plurality of oxygen saturation levels;
a communication circuit for transmitting a signal associated with at least one of the plurality of oxygen saturation levels measured by the sensor;
a controller for comparing at least one of the plurality of oxygen saturation levels measured by the sensor to a predetermined threshold oxygen saturation level; and
a remote device configured to receive the signal transmitted by the communication circuit and for providing an alarm in response to an undesired oxygen saturation level of which is relatively lower than the predetermined threshold oxygen saturation level.

2. The system of claim 1, wherein the sensor removably attaches to a garment.

3. The system of claim 2, wherein the sensor comprises a garment-mounted sensor.

4. The system of claim 2, wherein the garment comprises a pair of footed pajamas, a glove, or a hat.

5. The system of claim 1, wherein the sensor comprises a garment-integrated sensor.

6. The system of claim 1, wherein the sensor comprises a pulse oximetry sensor.

7. The system of claim 1, wherein the alarm comprises an audible alarm or a visual alarm.

8. The system of claim 1, wherein the signal comprises a single alarm signal or a stream of oxygen saturation levels.

9. The system of claim 1, wherein the controller is coupled with the sensor and operates the communication circuit to transmit the signal in response to identifying the undesired oxygen saturation level.

10. The system of claim 1, wherein the controller is coupled with the remote device and receives the plurality of oxygen saturation levels from the communication circuit via the signal.

11. The system of claim 10, wherein the communication circuit couples directly with the sensor.

12. The system of claim 1, wherein the communication circuit comprises a wireless transmitter.

13. The system of claim 1, wherein the remote device comprises a Smartphone, a tablet or an alarm clock.

14. A low blood oxygen level warning system, comprising:
a garment-integrated sensor for measuring a plurality of oxygen saturation levels;
a wireless transmitter for transmitting a signal associated with at least one of the plurality of oxygen saturation levels measured by the garment-integrated sensor;
a microcontroller for comparing the plurality of oxygen saturation levels measured by the garment-integrated sensor to a predetermined threshold oxygen saturation level, wherein the microcontroller is coupled with the garment-integrated sensor and operates the wireless transmitter to transmit the signal in response to identifying an undesired oxygen saturation level; and
a remote device configured to receive the signal transmitted by the wireless transmitter and for providing an alarm in response to the undesired oxygen saturation level of which is relatively lower than the predetermined threshold oxygen saturation level.

15. The system of claim 14, wherein the remote device comprises a Smartphone, a tablet or an alarm clock and the garment comprises a pair of footed pajamas, a glove, or a hat.

16. The system of claim 14, wherein the garment-integrated sensor comprises a pulse oximetry sensor.

17. The system of claim 14, wherein the alarm comprises an audible alarm or a visual alarm and the signal comprises a single alarm signal or a stream of oxygen saturation levels.

18. A method for activating an alarm in response to an undesired oxygen saturation level, comprising the steps of:
reading a plurality of oxygen saturation levels with a sensor;
comparing each of the plurality oxygen saturation levels read by the sensor to a predetermined safe oxygen saturation level;
transmitting a signal associated with at least one of the plurality oxygen saturation levels read by the sensor to a remote device over a communication network;
determining whether at least one of the plurality of oxygen saturation levels comprises the undesired oxygen saturation level, said undesired oxygen saturation level comprising an oxygen saturation level relatively lower than a predetermined threshold oxygen saturation level; and
activating the alarm in response to determining that one of the plurality of oxygen saturation levels comprises the undesired oxygen saturation level.

19. The method of claim 18, wherein the communication network comprises a wireless communication network.

20. The method of claim 18, including transmitting a warning signal to the remote device over the communication network.

21. The method of claim 18, including the step of streaming the plurality oxygen saturation levels to the remote device in real-time.

22. The method of claim 18, including the step of synching the alarm across multiple remote devices.

23. The method of claim 18, including the step of removably attaching the sensor to a garment.

24. The method of claim 18, including the step of sliding the sensor over an appendage of the user.

25. The method of claim 24, wherein the step of sliding the sensor over an appendage of the user further comprises the step of sliding the sensor over a toe, a finger, or an ear lobe of the user.

26. The method of claim 18, including the step of modifying the predetermined threshold oxygen saturation level.
